(19) United States
(12) Patent Application Publication
(10) Pub. No.: US 2020/0376187 A1
(43) Pub. Date: Dec. 3, 2020

(54) DUAL-TUBE DEVICE AN INFLATABLE CUFF FOR REMOVING IMPACTED FECAL MATTER FROM THE COLON, AND A METHOD OF USING THE SAME

(71) Applicant: Sean Devin Walcott, Jamaica, NY (US)

(72) Inventor: Sean Devin Walcott, Jamaica, NY (US)

(21) Appl. No.: 16/425,135

(22) Filed: May 29, 2019

(51) Int. Cl. A61M 3/02; A61M 1/00; A61M 25/10

(52) U.S. Cl. CPC A61M 3/0233; A61M 3/0279; A61M 1/0058; A61M 25/1002; A61M 2250/00; A61M 2025/1075; A61M 2202/0014; A61M 2202/068; A61M 2025/109

(57) ABSTRACT

A dual-tube removal device for treating obstipation of animals is provided. The first tube, or lubricant conduit, selectively channels lubricant or enema solution through dispersal ports provided near the distal end of the lubricant conduit. The second tube, or pilot tube, aids is selectively inflating an inflatable cuff adjacent the same distal end. In the inflated condition, with the infusion of lubricant and or enema solution and after guiding the distal end past the fecal blockage, a user may urge the fecal blockage out of the colon of the animal.

### BACKGROUND OF THE INVENTION

The present invention relates to medical devices to relieve obstipation and, more particularly, to a narrow, flexible, dual-tube device with an inflatable cuff along a distal end used for removing impacted fecal matter from the colon, and a method of using the same.

Obstipation or impacted fecal matter in pets and humans is a serious and life threating condition. This condition is secondary to pets being chronically dehydrated, various digestive conditions, even arthritis in pets can cause them to hold their bowls longer due to fear of posturing to defecate. Obstipation in pets such as dogs can be defined as advanced constipation and is associated the inability to pass the accumulation of dry hard feces. A large amount of hardened stool builds up in the colon of the pet causing abdominal discomfort, lack of appetite, lethargy, and vomiting, which can lead to organ failure and eventually death.

Currently obstipation is treated through the following: upon arrival at the veterinarian, the animal is sedated and the veterinarian then uses an enema solution or lubricant to aide in the removal of the impacted fecal matter. These enemas or lubrications are introduced into the colon via the rectal opening. Veterinarians use a French red rubber to infuse lubrications and or enema solution deep into the colon, at this point the French red rubber is removed and the veterinarian proceeds to use his/her index finger to reach into the colon and manually remove whatever fecal matter is within reach. Simultaneously, the veterinarian is deeply palpating the pet's abdomen in efforts to break apart the blockage in the colon and remove it piece by piece. This deep abdominal palpation can cause prolonged abdominal pain and the veterinarian using his/her finger can cause irritation and inflammation of the anus. Because the veterinarian is only using a finger this procedure can be lengthy, requiring prolonged sedation, wherein repeated attempts are often needed to clear the blockage.

The French red rubber that is used to de-obstipate colon does not adequately disperse lubrications/enema due to having only a few eyelets located at the last 1/8th of the tube. Also, it does not allow for any means of capturing or urging the blockage.

Furthermore, with the current method of de-obstipation, the veterinarian just can't reach far enough into the colon to remove the blockage in a timely manner, if at all. Also, there is the abdominal and rectal trauma form repeated attempts.

As can be seen, there is a need for a narrow, flexible, durable dual-tube tool with an inflatable cuff used for removing impacted fecal matter from the colon, and a method of using the same. The first tube provides lubricant and/or enema solution through a sufficient amount of ports for maximum lubricant/enema dispersal, while the second tube inflates the inflatable cuff/bladder above the blockage. This will allow the veterinarian to gain purchase, and with adequate lubrication in place slowly, pull the device out with all or large amounts of the blockage. The present invention minimizes the risks associated with sedation time, rectal trauma, abdominal trauma, while maximizing the efforts of user to improve the quality of life for the treated animal.

### SUMMARY OF THE INVENTION

In one aspect of the present invention, a dual-tube removal device for relieving obstipation includes the following: an elongated lubricant conduit having a distal end providing a plurality of dispersal ports; a first urging device operatively associate with a proximal end of the lubricant conduit; a pilot tube joined in parallel to a substantial portion of a length of the lubricant conduit; an inflatable cuff fluidly connected to a distal end of the pilot tube, wherein the inflatable cuff receives the distal end of the lubricant conduit; and a pilot urging device operatively associated with a proximal end of the pilot tube for urging the inflatable cuff to an inflated condition, wherein the inflatable cuff is located between one or more dispersal ports of the plurality of dispersal ports.

In another aspect of the present invention, a method of de-obstipating an animal with a fecal blockage includes the following: providing the above-mentioned dual-tube removal device for relieving obstipation; inserting the distal ends of the lubricant conduit and the pilot tubes in a colon of the animal so that the inflatable cuff is above the fecal blockage; infusing lubricant or enema solutions through the plurality of dispersal ports by way of the first urging device; inflating the inflatable cuff to the inflated condition by way of the pilot urging device; and removing said distal ends from said colon with the inflated cuff in the inflated condition.

These and other features, aspects and advantages of the present invention will become better understood with reference to the following drawings, description and claims.

### BRIEF DESCRIPTION OF THE DRAWINGS

FIG. 1 is a perspective view of an exemplary embodiment of the present invention;

FIG. 2 is an exploded view of an exemplary embodiment of the present invention;

FIG. 3 is a section view of an exemplary embodiment of the present invention, taken along line 3-3 of FIG. 1;

FIG. 4 is a detail section view of an exemplary embodiment of the present invention illustrating the lubricant tube and inflatable cuff in a deflated condition;

FIG. 5 is a detail section view of an exemplary embodiment of the present invention illustrating the lubricant tube and inflatable cuff in an inflated condition;

FIG. 6 is a schematic view of an exemplary embodiment of the present invention;

FIG. 7 is a schematic view of an exemplary embodiment of the present invention;

FIG. 8 is a perspective view of an exemplary embodiment of the present invention;

FIG. 9 is an exploded view of an exemplary embodiment of the present invention;

FIG. 10 is a section view of an exemplary embodiment of the present invention, taken along line 10-10 of FIG. 8;

FIG. 11 is a detail section view of an exemplary embodiment of the present invention illustrating the lubricant tube and inflatable cuff in a deflated condition;

FIG. 12 is a detail section view of an exemplary embodiment of the present invention illustrating the lubricant tube and inflatable cuff in an inflated condition; and

FIG. 13 is a flow chart of an exemplary embodiment of the present invention.

### DETAILED DESCRIPTION OF THE INVENTION

The following detailed description is of the best currently contemplated modes of carrying out exemplary embodiments of the invention. The description is not to be taken in a limiting sense, but is made merely for the purpose of illustrating the general principles of the invention, since the scope of the invention is best defined by the appended claims.

Broadly, an embodiment of the present invention provides a dual-tube removal device for treating obstipation of animals. The first tube, or lubricant conduit, selectively channels lubricant or enema solution through dispersal ports provided near the distal end of the lubricant conduit. The second tube, or pilot tube, aids is selectively inflating an inflatable cuff adjacent the same distal end. In the inflated condition, with the infusion of lubricant and or enema solution and after guiding the distal end past the fecal blockage, a user may urge the fecal blockage out of the colon of the animal.

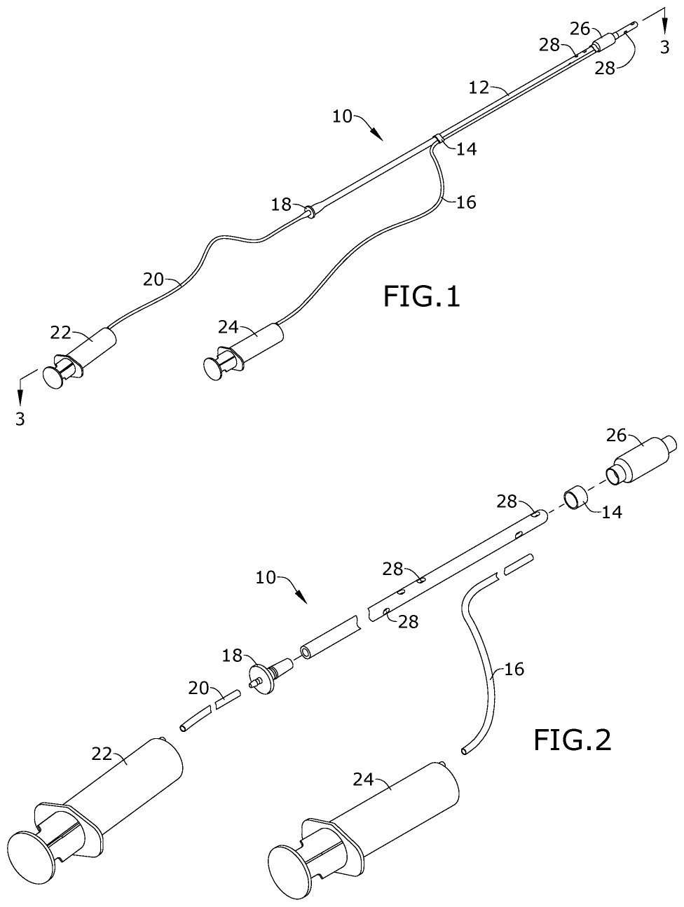

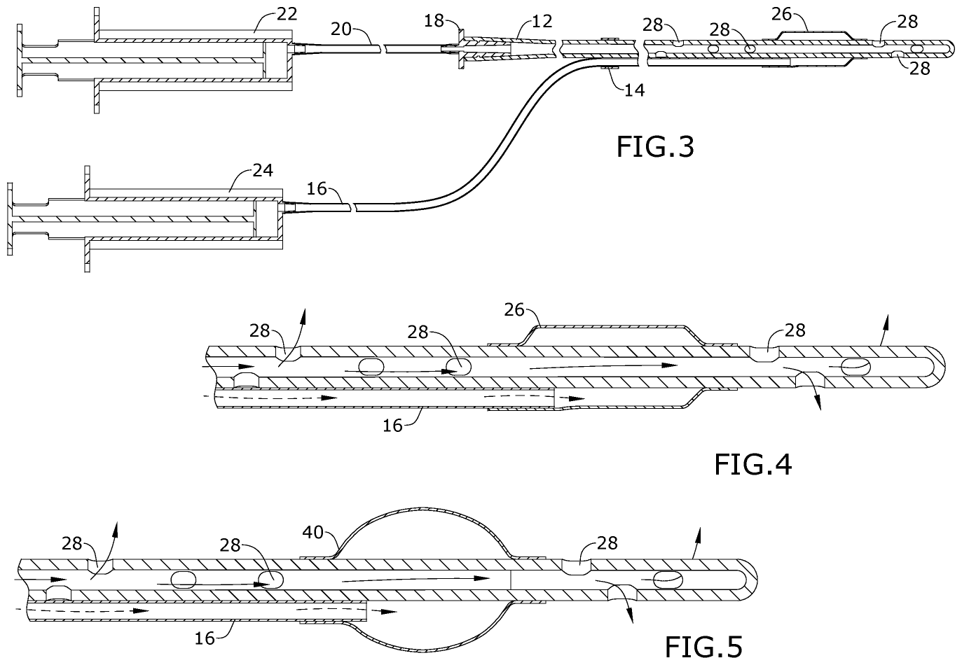

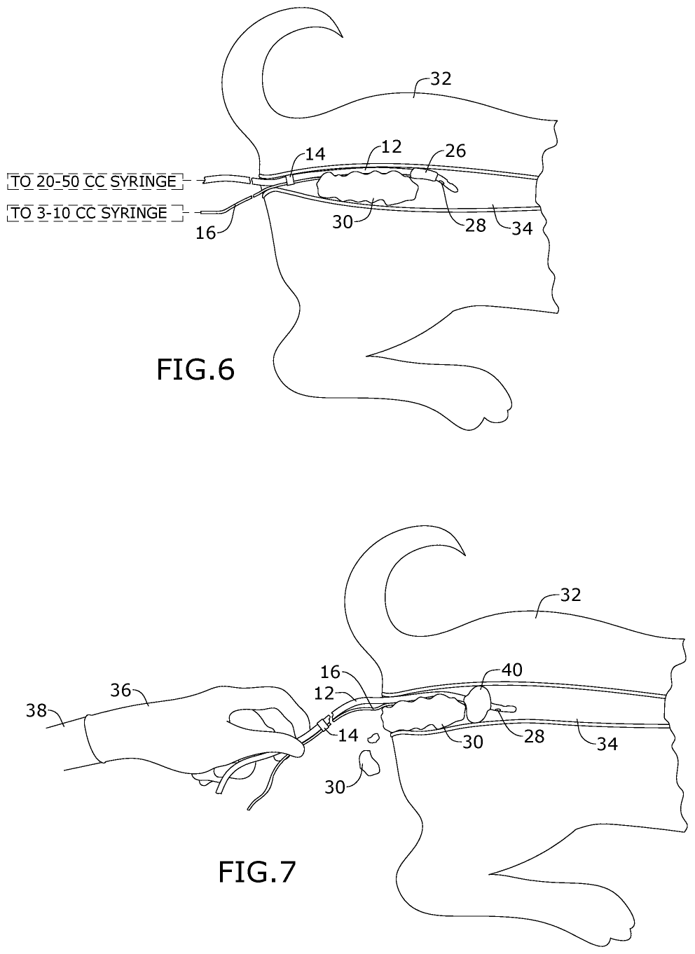

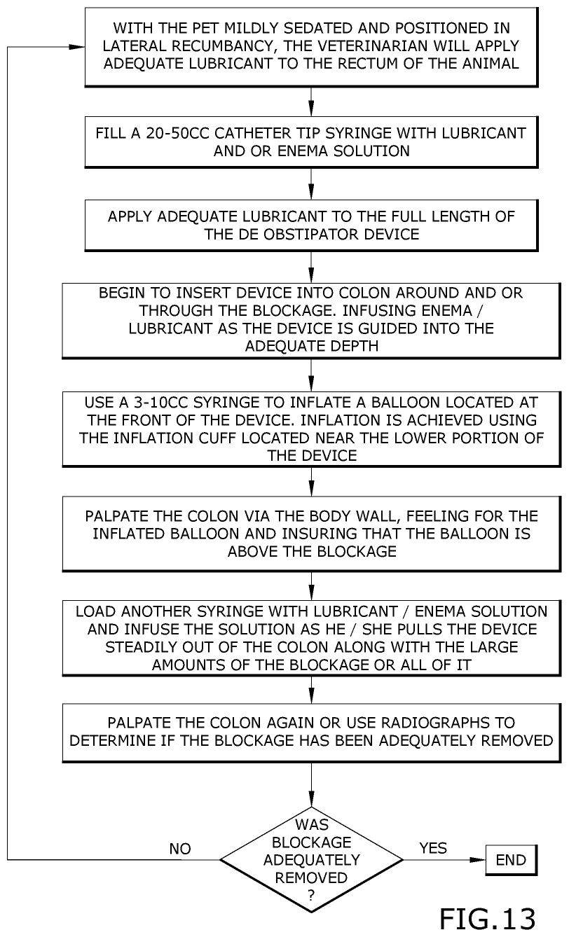

Referring to FIGS. 1 through 13, the present invention may include a method of de-obstipating a colon. The present invention embodies a dual-tube removal device 10 approximately 14-16 inches long and 3.0 mm diameter (long tube), air line is 1.6 mm diameter. The dual-tube removal device 10 includes a primary lubricant conduit running in parallel with a pilot tube.

In one embodiment, the elongpated primary lubricant conduit 12 may be fluidly coupled to an elongated first tube 20 by way of a luer 18 so that a first urging device 22, such as a first syringe, as illustrated in FIGS. 1-5, can urge fluid, such as lubricant or enema solution, through the first tube 10 into the primary lubricant conduit 12.

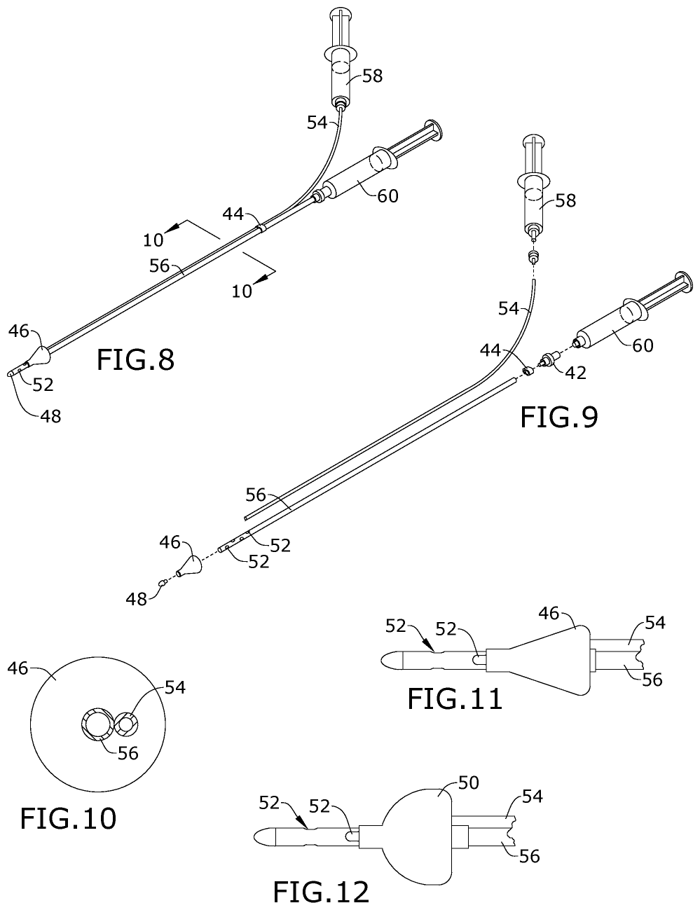

In another embodiment, the primary lubricant conduit 56 may be fluidly coupled by way of a luer 42 directly to the first urging device 60 for urging fluid, through the primary lubricant conduit 56, as illustrated in FIGS. 8-12. The distal end of the primary lubricant conduit 12 or 56 may provide dispersal ports 28 or 52, respectively, so that urged lubricant/enema solution can fluidly disperse therethrough.

Operatively associated with the primary lubricant conduit 26 or 46 may be a pilot tube 16 or 54, respectively, wherein said pilot tube 16 or 54 is physically though removably joined along a substantial length of the primary lubricant conduit 26 or 50; in certain embodiments, by way of a rubber tie 14 or 44 or the like. The pilot tube 16 or 54 is fluidly coupled to a pilot urging device 24 or 58, such as a second syringe or manual bulb-style pump, for urging air through the respective pilot tube 16 or 54. An inflatable cuff or bladder 26 or 46 may be fluidly coupled to the distal end of the pilot tube 16 or 54, respectively, for moving between a deflated condition and an inflated condition. The pilot tube 16 or 54 may provide a relief valve at the proximal end.

The inflatable cuff 26 or 46 provides a lumen for slidably receiving the primary lubricant tube 12 so as to be disposed proximal of the distal end of said primary lubricant tube 12, as illustrated in FIG. 1, so that the inflatable cuff 26, 46 or 50 is located between a plurality of dispersing ports—in other words, lubricant urged through the primary lubricant tube 12 can be dispensed before/below and beyond/above the inflatable cuff 26 or 46. The inflatable cuff may be cylindrical 26, pyramidal tapered 46, bulbous, or the like as long as it can function as disclosed herein. In an alternative embodiment, a conduit tip 48 may be provided at the very distal end of the primary lubricant tube 56. The components used in the present invention may be made of rubberized plastics using injection, extrusion and/or blow molding processes.

A method of using the present invention may include the following. The dual-tube removal device 10 disclosed above may be provided. A veterinarian-user 38 with medical gloves 36 on and with an animal 32 mildly sedated and positioned in lateral recumbancy, may apply adequate lubricant to the rectum of animal 32. Then the user may fill the first urging device 22, which may be a 20-50 cc catheter tip syringe, with lubricant and or enema solution. The user may then apply adequate lubricant to the full length of the exterior of the dual-tube removal device/de-obstipator device 10. Then the user would begin to insert the dual-tube removal device 10 into the colon 34 around and or through blockage 30, infusing enema/lubricant as the dual-tube removal device 10 is guided in to adequate depth. At this point the veterinarian-user 38 may use the pilot urging device 24 or 58, such as a 3-10 cc syringe, to inflate the inflatable cuff 26 or 46 located at the front portion of the dual-tube removal device 10 to the inflated condition 40 or 50, respectively; the. inflation is achieved using the pilot urging device 24 or 58 located near the lower portion of the dual-tube removal device 10 where the user 38 is working. At this point the user 38 may palpate the colon 34 via the body wall, feeling for the inflatable cuff 26 or 46 in the inflated condition and ensuring that the inflatable cuff 26 or 46 is above the blockage 30, as illustrated in FIG. 7. Lastly, the user 38 may load another predetermined amount with lubricant/enema solution and infuse the solution through the dispersal ports 28 or 52 as he/she pulls the dual-tube removal device 10 steadily out of the colon along with large amounts of the blockage or all of it. The veterinarian 38 may then palpate the colon 34 again or use radiographs to determine if the blockage 30 has been adequately removed.

This procedure is almost always successful and eliminates the need for sedation and surgery for most animals 32. The use of the dual-tube removal device 10 provides for the treatment and relief of obstipation in pets in a manner which is not only quick, easy and effective, but safe as well. The relatively simple design of the present invention and the material of construction make the dual-tube removal device 10 a cost-effective design due to the relatively low material and labor costs involved. Final production of the dual-tube removal device 10 may be performed by manufacturing workers of average skill.

Additionally, the present invention may be used in cases of impacted fecal material for people; specifically, the elderly and infants.

It should be understood, of course, that the foregoing relates to exemplary embodiments of the invention and that modifications may be made without departing from the spirit and scope of the invention as set forth in the following claims.

What is claimed is:

1. A dual-tube removal device for relieving obstipation, comprising:
an elongated lubricant conduit having a distal end providing a plurality of dispersal ports;
a first urging device operatively associate with a proximal end of the lubricant conduit;
a pilot tube joined in parallel to a substantial portion of a length of the lubricant conduit;
an inflatable cuff fluidly connected to a distal end of the pilot tube, wherein the inflatable cuff receives the distal end of the lubricant conduit; and
a pilot urging device operatively associated with a proximal end of the pilot tube for urging the inflatable cuff to an inflated condition.

2. The dual-tube removal device for relieving obstipation of claim 1, wherein the inflatable cuff is located between one or more dispersal ports of the plurality of dispersal ports.

3. The dual-tube removal device for relieving obstipation of claim 1, further comprising a tie that joins the pilot tube and the lubricant conduit in parallel.

4. The dual-tube removal device for relieving obstipation of claim 1, wherein the first urging device is a 20 to 50 cc syringe.

5. The dual-tube removal device for relieving obstipation of claim 1, wherein the pilot urging device is a 3 to 10 cc syringe.

6. The dual-tube removal device for relieving obstipation of claim 1, wherein the inflatable cuff is cylindrical.

7. The dual-tube removal device for relieving obstipation of claim 1, wherein the inflatable cuff is pyramidal.

8. A method of de-obstipating an animal with a fecal blockage, comprising:
providing the dual-tube removal device for relieving obstipation of claim 2;
inserting the distal ends of the lubricant conduit and the pilot tubes in a colon of the animal so that the inflatable cuff is above the fecal blockage;
infusing lubricant or enema solutions through the plurality of dispersal ports by way of the first urging device;
inflating the inflatable cuff to the inflated condition by way of the pilot urging device; and
removing said distal ends from said colon with the inflated cuff in the inflated condition.
